(19) United States
(12) Patent Application Publication
(10) Pub. No.: US 2026/0104421 A1
(43) Pub. Date: Apr. 16, 2026

(54) CONNECTOR, MARKER, CAPTURE CONSTRUCT AND DATA STORAGE DEVICE

(71) Applicant: LEICA MICROSYSTEMS CMS GMBH, Wetzlar (DE)

(72) Inventor: Soeren ALSHEIMER, Wetzlar (DE)

(21) Appl. No.: 19/105,051

(22) PCT Filed: Nov. 18, 2022
(86) PCT No.: PCT/EP2022/082452

§ 371 Date: Feb. 20, 2025

(30) Foreign Application Priority Data
Aug. 23, 2022 (EP) 22191689.3
Aug. 23, 2022 (EP) 22191690.1

(51) Int. Cl. G01N 33/58; G01N 33/532

(52) U.S. Cl. CPC G01N 33/58; G01N 33/532; G01N 2458/10

(57) ABSTRACT

A connector for generating a biological structure includes a protein backbone, a first reactive interactor arranged towards a first end of the protein backbone and configured to covalently bind to a second reactive interactor, and a first affinity interactor arranged towards a second end of the protein backbone and configured to bind to a second affinity interactor. The protein backbone includes a cleavage site between the first end and the second end of the protein backbone. One of the first reactive interactor and the first affinity interactor is configured to bind to a first affinity reagent including the second reactive interactor or the second affinity interactor. Another one of the first reactive interactor and the first affinity interactor is configured to bind to a label or a second affinity reagent comprising the second reactive interactor or the second affinity interactor.

### CROSS REFERENCE TO RELATED APPLICATIONS

This application is a U.S. National Phase application under 35 U.S. C. § 371 of International Application No. PCT/EP2022/082452, filed on Nov. 18, 2022, and claims benefit to European Patent Application No. EP 22191690.1, filed on Aug. 23, 2022 and European Patent Application No. EP 22191689.3, filed on Aug. 23, 2022. The International Application was published in English on Feb. 29, 2024 as WO 2024/041748 A1 under PCT Article 21(2).

### FIELD

Embodiments of the invention relate to a connector for a marker, a capture construct or a data storage device and methods and device to generate a marker, a capture construct or a data storage device.

### BACKGROUND

The generation of structures from biological components or building blocks has increasingly come into focus. Especially the high specificity and high affinity of biological components such as antibodies or oligonucleotides are properties that may be exploited when building biological structures from these such as markers.

As a particular example: Spatial biology is an emerging field of microscopy wherein different strategies are used to image biological samples, for example tissue sections, with high spatial resolution and whilst analysing a high number of markers. The number of markers necessary in this application may be anywhere ranging from around 100 up to 10,000s in order to mark a high number of targets in a sample. This necessitates a cyclical staining, imaging, blanking process as well as the construction of large libraries of markers prior to their use. In contrast to most of present-day microscopic assays for example immunofluorescence or FISH experiments, which involve only a handful of markers, these new approaches require significantly more time to stain samples, image samples, as well as process and analyse data. Furthermore, they require significantly more valuable reagents, such as markers.

A particular problem remains the rapid and efficient generation of large libraries of biological structures, such as markers, with a variety of properties.

### SUMMARY

Embodiments of the present invention provide a connector for generating a biological structure. The connector includes a protein backbone, a first reactive interactor arranged towards a first end of the protein backbone and configured to covalently bind to a second reactive interactor, and a first affinity interactor arranged towards a second end of the protein backbone and configured to bind to a second affinity interactor. The protein backbone includes a cleavage site between the first end and the second end of the protein backbone. One of the first reactive interactor and the first affinity interactor is configured to bind to a first affinity reagent including the second reactive interactor or the second affinity interactor. Another one of the first reactive interactor and the first affinity interactor is configured to bind to a label or a second affinity reagent comprising the second reactive interactor or the second affinity interactor.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view of connectors in states before and after cleaving of a cleavage site according to some embodiments;

FIG. 2 is a schematic view of several configurations of streptavidin according to some embodiments;

FIG. 3 is a schematic view of the nucleic acid backbone according to FIG. 2 with sequencing information of attached target nucleic acid strands, according to some embodiments;

FIG. 4 is a schematic view of a label comprising a second reactive interactor, according to some embodiments;

FIG. 5 is a schematic view of steps to assemble a marker according to some embodiments;

FIG. 6 is a schematic view of steps to assemble a data storage device according to some embodiments; and

FIG. 7 is a schematic view of steps to assemble a capture construct according to some embodiments.

### DETAILED DESCRIPTION

Embodiments of the present invention provide building blocks for efficiently generating biological structures.

A connector is provided for generating, in particularly assembling, a biological structure comprising: a protein backbone; a first reactive interactor arranged towards or at a first end of the protein backbone and configured to covalently and specifically bind to a second reactive interactor; and a first affinity interactor arranged towards or at a second end of the protein backbone and configured to non-covalently and specifically bind to a second affinity interactor. The protein backbone comprises a cleavage site between the first end and the second end of the protein backbone. Further, one of the first reactive interactor and the first affinity interactor is configured to bind to a first affinity reagent comprising the respective second reactive interactor or second affinity interactor, and the other one of the first reactive interactor and the first affinity interactor is configured to bind to a label or a second affinity reagent comprising the respective second reactive interactor or second affinity interactor.

The connector allows two molecular and/or biological building blocks to be selectively linked to each other in an efficient way, which preferably leads to linkages with practically no spontaneous dissociation yet is compatible with the introduction of cleavage sites that allow the cleaving of the connection at the user's discretion. The connector with both pairs of interactors may in particular be used to enable the rapid, cost-efficient production of large libraries of markers, wherein a marker is the conjugate of a label and an affinity reagent and wherein a label is a construct bearing one or more labeling agents, like a fluorescent dye or a combination of fluorescent dyes.

Preferably the backbone is cleavable specifically at the cleavage site by an enzyme, in particular a protease, by cleavage light, or by a temperature change. This enables flexible separation of the ends of the connector, for example, when using the connector in methods with iterative steps, in which the repeated removal of parts of the connector is required.

Preferably, the first reactive interactor and the second reactive interactor are configured to form a bioconjugate. For example, the reactive interactor pair may be SpyTag/SpyCatcher. This enables high specificity of the reactive interactor pair and therefore a particular robust covalent binding of the reactive interactors.

Preferably, the first affinity interactor is one of streptavidin and biotin and the second affinity interactor is the other one of streptavidin and biotin. This enables high affinity and high specificity of the affinity interactor pair and therefore a robust binding of the affinity interactors.

Preferably, the streptavidin is a tetramer with one active biotin binding site or a monomer with one active biotin binding site. This enables binding of the affinity interactor pair at a predictable stoichiometric ratio.

Preferably the connector is a fusion protein including the first affinity interactor, the protein backbone, the cleavage site and the first affinity interactor. This enables particularly efficient production of the connector.

Preferably, the first affinity interactor is streptavidin and the first affinity reagent is an oligonucleotide comprising the biotin. In particular, the first affinity reagent is a biotinylated oligonucleotide. This enable a particular easy production of the first affinity reagent.

Preferably the connector comprises the first affinity reagent. In particular, the first affinity reagent being the biotinylated oligonucleotide configured to bind with high affinity to the streptavidin of the protein backbone.

In another aspect, a marker for analysing biological samples is provided, comprising: the label, in particular comprising at least a dye, bound to the connector of above. The marker may be attached to a target analyte in a biological sample by means of the first affinity reagent, for example. The marker is particular efficient to generate from the individual building blocks such as the connector, the label and the first affinity reagent. Moreover, a large variety of markers may be generated from these individual building blocks having different properties. For example, label may be used having different optical properties in order to efficiently generate markers with these different optical properties.

Preferably, the label comprises the second reactive interactor covalently bound to the first reactive interactor of the connector, in particular, forming a bioconjugate. This enables particularly robust connection of the label to the connector.

Preferably, the marker comprises a third affinity reagent, the third affinity reagent comprising an oligonucleotide at least partially complementary to the first affinity reagent, in particular a biotinylated oligonucleotide, of the connector and hybridised to the first affinity reagent. Thus, either the biotinylated oligonucleotide or the third affinity reagent may bind to a target molecule in a biological sample.

Preferably, the third affinity reagent further comprises an antibody. The antibody may bind to a target molecule in a biological sample. This enables binding to a large variety of target molecules with particularly high specificity.

Preferably, the marker comprises a base oligonucleotide comprising the second reactive interactor and at least one oligonucleotide conjugated dye hybridised to the base oligonucleotide via complementary base pairing. This enables labels that may be generated particularly efficiently.

In a further aspect a capture construct for capturing analytes of a biological sample is provided, comprising: a DNA-origami backbone with at least a capture region; at least one affinity capture reagent comprising the connector of above and the second affinity reagent. The first affinity reagent of the connector binds the affinity capture reagent to the capture region of the DNA-origami backbone and the first affinity reagent is a staple strand of the DNA-origami backbone, preferably comprising the second affinity interactor. Further, the second affinity reagent is configured to capture one of the analytes of the biological sample, and preferably the second affinity reagent comprises the second reactive interactor and is covalently bound to the first reactive interactor of the connector. The capture construct enables capturing the plurality of analytes at predetermined positions, the capture regions, on the backbone. Further, the capture construct enables capturing the analytes at a particularly high density. The capturing of the plurality of analytes enables subsequent analysis of the plurality of analytes.

Preferably, the DNA-origami backbone comprises at least a first orientation indicator and a second orientation indicator. The first orientation indicator and the second orientation indicator may be, for example, fluorescent dyes attached to the backbone. The orientation indicators enable determining the spatial orientation or the directionality of the capture construct, in particular the backbone. In this way, the orientation indicators enable spatial encoding. This means, different positions on the backbone may be assigned to capture regions that have reactivities to distinct analytes.

Preferably, the DNA-origami backbone comprises nucleic acids. These DNA origami structures may range in size from a few nanometres into the micron range. For the fabrication of such DNA origami-based structures longer DNA molecules (scaffold strands) are folded at precisely identified positions by so called staple strands. The DNA origami may be designed to provide a self-assembly backbone of a particular predetermined shape. This enables an easy and reproducible synthesis and assembly of the backbone. Staple strands may be position-selectively functionalised. The positional resolution in this case is limited by the size of a nucleotide, which is in the range of a nanometre or below. This has been exploited in the prior art to generate fluorescent standards, wherein fluorescent dyes are connected to precisely located bands on the DNA origami. These standards are known as “nanoruler” and are used for the calibration of imaging systems like confocal or super resolution microscopes (e.g. STED), for example, as disclosed by US2014/0057805 A1.

The DNA origami backbone provides a scaffold for the affinity capture reagents. Preferably, the DNA origami structure comprises at least one scaffold strand and multiple staple strands, wherein the staple strands are complementary to at least parts of the scaffold strand and configured to bring the scaffold strand into a predetermined conformation. In particular, the strands are oligonucleotides. This enables generating backbones with predetermined two-or three-dimensional shapes that can self-assemble. Further, this enables the site-specific placement of capture regions on the backbone. Preferably, the affinity capture reagents of the capture regions may be attached to staple strands of the backbone at predetermined positions. Staple strands allow the spatially precise functionalisation of the DNA origami at their respective locations on the DNA origami. Thus, each capture region may be located along the backbone at a particular staple strand or group of staple strands that are in close proximity. Since the staple strands are located at predetermined positions the positions of the capture regions may equally be predetermined.

Further details of capture constructs are disclosed in application PCT/EP2022/058640. The content of which is incorporated herein by reference in its entirety.

In a further aspect a data storage device comprising is provided, comprising: a DNA-origami backbone with a plurality of attachment sites, the label, in particularly comprising at least a dye, bound to the connector. The first affinity reagent is an oligonucleotide staple strand of the DNA-origami backbone, preferably comprising the second affinity interactor, and the first affinity reagent comprises a unique oligonucleotide sequence configured to bind the connector with the label to a complementary sequence of one of the attachment sites.

Preferably, the DNA-origami backbone of the data storage device comprises at least a first orientation indicator and a second orientation indicator. The first orientation indicator and the second orientation indicator may be, for example, fluorescent dyes attached to the backbone. The orientation indicators enable determining the spatial orientation or the directionality of the capture construct, in particular the backbone. In this way, the orientation indicators enable spatial encoding. This means, different positions on the backbone may be assigned to capture regions that have reactivities to distinct analytes.

The attachment sites being unique nucleic acid sequences, preferably of the staple strands. Preferably, the labels may be attached to staple strands of the backbone at predetermined attachment sites. Since the staple strands are located at predetermined positions the positions of the attachment sites may equally be predetermined.

By providing or generating the DNA-origami backbone in a particular or predetermined manner, the position of the attachment sites for the labels relative to the backbone and/or the at least one first orientation indicator and one second orientation indicator are predetermined or known. Thus, providing a suitable or predetermined plurality of labels to attach at the corresponding attachment sites, information can be stored at the data storage device.

Further details of data storage devices are disclosed in application EP22191690.1. The content of which is incorporated herein by reference in its entirety.

In a further aspect, a method for generating the marker is provided, comprising the following steps: combining the first affinity reagent with the connector, in particular binding the biotinylated oligonucleotide to the connector, and combining the connector with the label, in particular forming a bioconjugate.

Preferably, the method further comprises the following step: combining the first affinity reagent with the third affinity reagent. In particular, the first affinity reagent is the biotinylated oligonucleotide and the third affinity reagent is an oligonucleotide conjugated antibody having a partially complementary sequence to the biotinylated oligonucleotide. In this step, the biotinylated oligonucleotide binds to the oligonucleotide of the antibody.

In a further aspect, a method for generating the capture construct or for generating the data storage device is provided, comprising the following steps: combining the first affinity reagent with the connector, combining the connector with the label or the second affinity reagent, and combining the first affinity reagent with the DNA-origami backbone. This enables particularly efficient generation of the capture construct or the storage device.

In a further aspect, a device for generating a marker, a capture construct or a data storage device is provided, comprising: a liquid dispensing unit, a plurality of liquid containers, and a control unit configured to direct the liquid dispensing unit to dispense liquids containing labels, first affinity reagents, second affinity reagents, third affinity reagents, or DNA-origami backbones into the liquid containers according to the method steps above. This enables particularly efficient generation of the marker, the capture construct or the data storage device.

Preferably, the liquid dispensing unit is configured to dispense the liquids with acoustic droplet ejection. This enables particularly accurate dispensing of small volumes.

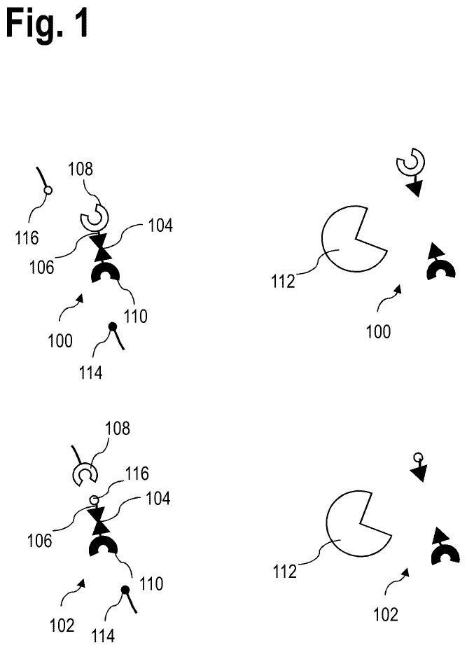

FIG. 1 is a schematic view of connectors 100, 102 in states before and after cleaving of a cleavage site 104 of the connectors 100, 102. The connectors 100, 102 comprise a protein backbone 106 that comprises the cleavage site 104. At a first end of the connectors 100, 102 a first reactive interactor 108 is arranged and at a second end of the connectors 100, 102 a first affinity interactor 110 is arranged. Preferably, both, the first affinity interactor 110 and the first reactive interactor 108, are a protein. Thus, the protein backbone 106 and the interactors 110, 108 may be generated by expression from a single expression cassette.

The cleavage site 104 may be a particular motif cleavable by a protease 112, for example. Alternatively, the cleavage site 104 may be light or temperature sensitive, such that the protein backbone 106 is cleaved by light or a temperature change. After cleaving of the cleavage site 104, the first end of the respective connector 100, 102 and the second end of the respective connector 100, 102 are separated from each other. This cleavage may be irreversible.

The first reactive interactor 108 is configured to irreversibly, covalently bind to a second reactive interactor 116. Examples of these pairs of reactive interactors 108, 116 include proteins that form bioconjugates such as Tag/Catcher pairs, for example, SpyTag/SpyCatcher. Thus, the first reactive interactor 108 may be the Tag part and the second reactive interactor 116 may be the Catcher part, for example, as in connector 100. However, alternatively the first reactive interactor 108 may be the Catcher part and the second reactive interactor 116 may be the Tag part as in connector 102.

The first affinity interactor 110 is configured to specifically bind to a second affinity interactor 114. The first and second affinity interactor 110, 114 specifically bind to each other based on intermolecular forces, such as ionic bonds, hydrogen bonds and Van der Waals forces and the binding is reversible. A preferred example of these pairs of affinity interactors 110, 114 is biotin and streptavidin that bind to each other with very high affinity. The binding of biotin to streptavidin in irreversible under physiological conditions. Similarly to the first and second reactive interactors 108, 116, the first affinity interactor 110 may be one of the biotin and streptavidin and the second affinity interactor 114 may be the other one of biotin and streptavidin.

Thus, the connectors 100, 102 are enabled to specifically binding to two further entities that either comprise one of the affinity interactors 110, 114 or one of the reactive interactors 108,116, whilst at the same time enabling subsequent separation by means of the cleavage site 104.

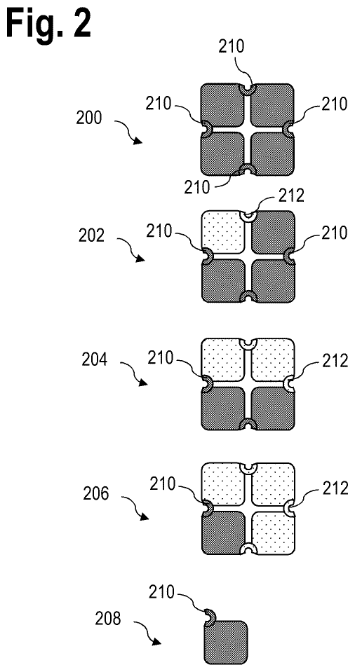

FIG. 2 is a schematic view of several configurations 200, 202, 204, 206, 208 of streptavidin. The first configuration 200 of streptavidin is a native tetramer with four active biotin binding sites 210. The second configuration 202 of streptavidin is a tetramer with three active sites 210 and one non-functional biotin binding site 212. The third configuration 204 of streptavidin is a tetramer with two active sites 210 and two non-functional biotin binding sites 212. The fourth configuration 206 of streptavidin is a tetramer with one active site 210 and three non-functional biotin binding site 212. The fifth configuration 208 of streptavidin is a monomer with one active site 210.

By varying the number of active sites 210, the number of entities able to bind to the connectors 100, 102 may be varied. Further, by choosing the configurations 206, 208 with only one binding site, the stoichiometric ratio of connectors 100, 102 to entities binding to each other via the first and second affinity interactors 110, 114 is predictable, such that only one connector 100, 102 binds to one entity.

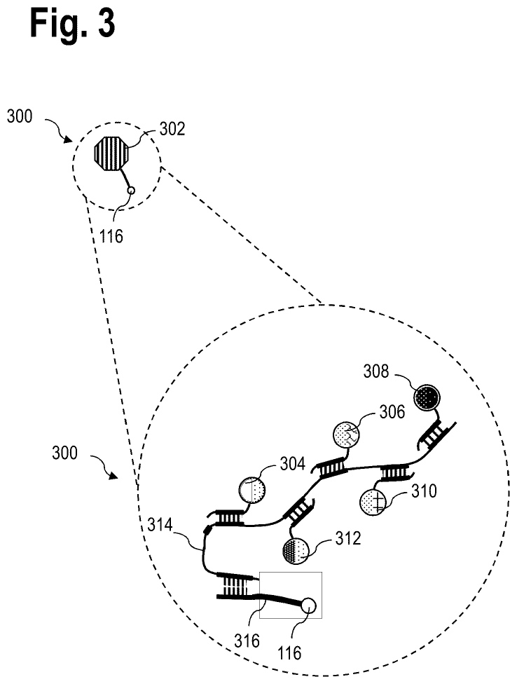

FIG. 3 is a schematic view of a label 300 comprising the second reactive interactor 116. This enables binding of the label 300 to the connector 100. Alternatively, the label 300 may comprise any one of the other of first reactive interactor 108, first or second affinity interactor 110, 114 in order to bind the label 300 to a connector comprising the matching interactor 108, 110, 114, 116 of the respective pair.

The label 300 comprises dyes, which is symbolised in FIG. 3 by the shape marked with the reference sign 302. The label 300 comprises five individual fluorescent dyes 304, 306, 308, 310, 312. These differ in their optical properties, such as excitation wavelength, emission wavelength and emission lifetime. The dyes 304, 306, 308, 310, 312 are each conjugated to oligonucleotides that are configured to hybridise to a particular sequence on a central nucleic acid backbone 314. The central nucleic acid backbone 314 is in turn configured to hybridise to a particular oligonucleotide 316 comprising the second reactive interactor 116.

The label 300 may be flexibly assembled from the individual parts 304, 306, 308, 310, 312, 314, 316. For example, in order to assemble a plurality of unique labels, a plurality of individual dyes 304, 306, 308, 310, 312 conjugated to oligonucleotides may be combined with the central nucleic acid backbone 314 and the oligonucleotide 316 comprising the second reactive interactor 116. The unique base-pairing between the individual oligonucleotides enables assembly of predetermined labels. For example, labels may differ in the optical properties of the individual dyes attached to the central nucleic acid backbone 314.

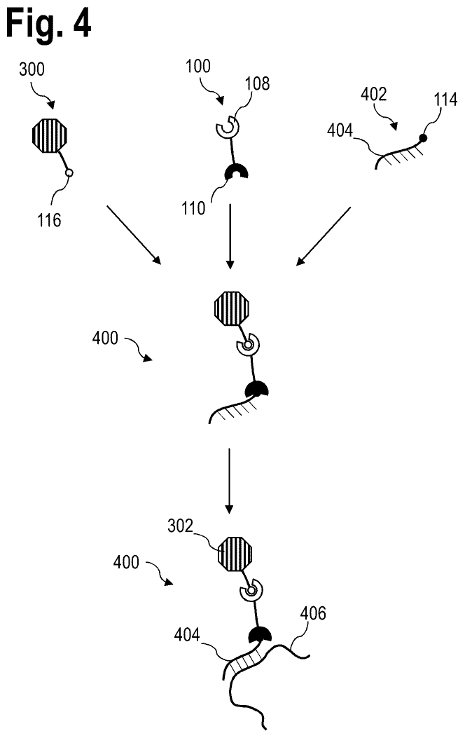

FIG. 4 is a schematic view of steps to assemble a marker 400. The marker 400 may be generated by combining the label 300 with the connector 100 and a first affinity reagent 402. The label 300 comprising the second reactive interactor 116 binds to the first reactive interactor 108 of the connector 100 to form a bioconjugate. The first affinity reagent 402 comprising the second affinity interactor 114 binds to the first affinity interactor 110 of the connector 100. The first affinity reagent 402 may, for example, be an oligonucleotide 404 with a biotin 114 moiety. Due to the specificity of the individual interactors 108, 110, 114, 116 to the respective opposite interactor 108, 110, 114, 116, the marker 400 may be assembled in an efficient manner.

The marker 400 may be introduced into a biological sample in order to mark a particular biological structure, based on the specific affinity of the first affinity reagent 402. For example, the first affinity reagent 404 of the marker 400 may mark a genetic structure 406 by binding to it by base-pairing. Thus, when detecting the fluorescence of the dye 302 in the sample, for example, by means of a microscope, the location of the dye 302 reveals the location of the structure 406 in the sample.

When assembling the marker 400, a mix-and-match approach may be adopted. For example, a particular label with particular optical properties, as described for FIG. 3, may efficiently connected to a particular first affinity reagent 402 that has an affinity for a particular biological structure in the biological sample. This way, simply by combining in a container the solutions with the particular label 300, the connector 100 and the particular first affinity reagent 402 a particular marker 400 may be generated. At the same time, a different marker may be generated by combining in a container the solutions with another label, the connector and another first affinity reagent, in particular with affinity to another biological structure in the biological sample. Thus, several different markers with individual optical properties and affinities to specific biological structures of the biological sample may be generated efficiently.

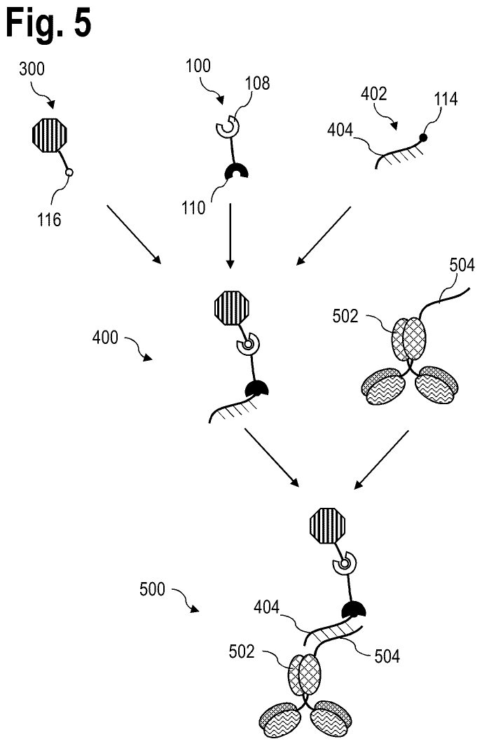

FIG. 5 is a schematic view of a marker 500 with a further affinity reagent 502. The further affinity reagent 502 may be an oligonucleotide 504 conjugated antibody. The marker 500 may be assembled by combining the marker 400 with the further affinity reagent 502 and the oligonucleotide 504 being configured to hybridise to the first affinity reagent 404. The marker 500 may then be used to mark that biological structure in a biological sample that the further affinity reagent 502 has an affinity to. As explained above for FIG. 4, by mixing-and-matching makers 400 with particular labels with further affinity reagent 502 a plurality of markers with specific affinities and optical properties may be assembled efficiently.

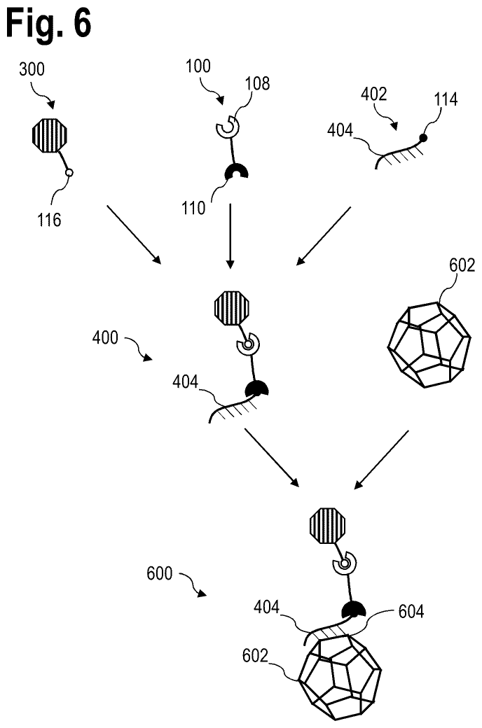

FIG. 6 is a schematic view of steps to assemble a data storage device 600. The data storage device 600 is assembled from the marker 400 and a DNA-origami backbone 602. The DNA-origami backbone 602 has a plurality of attachment sites 604. The marker 400, in particular the first affinity reagent 404 of the marker 400, may hybridise to one of the attachment sites 604 of the backbone 602. Each attachment site 604 of the backbone 602 has a unique oligonucleotide sequence and the first affinity reagent 404 of each marker 400 comprises a complementary oligonucleotide sequence. Thus, a plurality of markers, each with unique complementary sequences, may be attached to a particular backbone 602. By placing the markers at particular attachment sites and by using markers with different optical properties, information may be stored. The connector 100 enables assembling the data storage device 600 efficiently by assembling a variety of different markers 400 and combining them with the DNA-origami backbone 602.

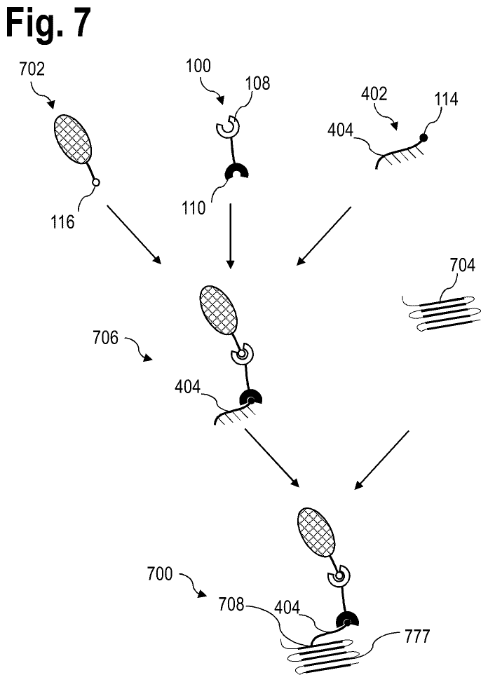

FIG. 7 is a schematic view of steps to assemble a capture construct 700. The capture construct comprises a second affinity reagent 702, the connector 100, the first affinity reagent 402 and a DNA-origami backbone 704. The capture construct 700 is assembled by combining the second affinity reagent 702, the connector 100, the first affinity reagent 402 in order to assemble an affinity capture reagent 706. As explained above, the respective interactors 108, 110, 114, 116 enable the efficient assembly of the individual parts. The capture construct 700 comprises at least one capture region 708, to which the affinity capture reagent 706, in particular the first affinity reagent 404, binds. The capture region 708 may comprise a plurality of the affinity capture reagents 706. The second affinity reagent 702 may be a nanobody, for example, with affinity to a particular target analyte of a biological sample and comprise the second reactive interactor 116. Thus, when adding the capture construct 700 to the biological sample, the target analyte is captured by the capture construct 700, for example, in order to subject the target analyte to further analysis. The connector 100 enables assembling the capture construct 700 efficiently by efficiently assembling a variety of different affinity capture reagents 706 and binding them to individual capture regions 708 of the DNA-origami backbone 704. The capture regions 708 may differ from each other in that the respective affinity capture reagents 706 have second affinity reagents 702 that have affinity to a particular target analyte.

In order to assemble the marker 400, 500 the data storage device 600, or the capture construct 700, each of their respective individual components may be provided in a liquid solution and by combining the solutions, for example by means of a liquid dispensing unit, the respective interactors 108, 110, 114, 116 of the connector 100, 102 bind the components together. By mixing-and-matching different first and second affinity reagents, that means by combining the solutions of different first and second affinity reagents, a plurality of markers 400, 500 the data storage devices 600, or the capture constructs 700 may be efficiently assembled from the individual components.

Identical or similarly acting elements are designated with the same reference signs in all Figures. As used herein the term “and/or” includes any and all combinations of one or more of the associated listed items and may be abbreviated as “/”. Individual features of the embodiments and all combinations of individual features of the embodiments among each other as well as in combination with individual features or feature groups of the preceding description and/or claims are considered disclosed.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

100,102 Connector
104 Cleavage site
106 Protein backbone
108 First reactive interactor
110 First affinity interactor
112 Protease
114 Second affinity interactor
116 Second reactive interactor
200, 202, 204, 206, 208 Streptavidin configurations
210 Functional biotin binding site
212 Non-functional biotin binding site
300 Label
302 Dye
304, 306, 308, 310, 312 Fluorescent dye
314 Central nucleic acid backbone
316 Oligonucleotide of label
400, 500 Marker
402 First affinity reagent
404 Oligonucleotide of first affinity reagent
406 Genetic structure
502 Antibody
504 Oligonucleotide of antibody
600 Data storage device
602, 704 DNA-origami backbone
604 Attachment site
700 Capture construct
702 Second affinity reagent
706 Affinity capture reagent
708 Capture region

1. A connector for generating a biological structure, the connector comprising:
a protein backbone,
a first reactive interactor arranged towards a first end of the protein backbone and configured to covalently bind to a second reactive interactor, and
a first affinity interactor arranged towards a second end of the protein backbone and configured to bind to a second affinity interactor,
wherein the protein backbone comprises a cleavage site between the first end and the second end of the protein backbone
wherein one of the first reactive interactor and the first affinity interactor is configured to bind to a first affinity reagent comprising the second reactive interactor or the second affinity interactor, and
wherein another one of the first reactive interactor and the first affinity interactor is configured to bind to a label or a second affinity reagent comprising the second reactive interactor or the second affinity interactor

2. The connector according to claim 1, wherein the backbone is cleavable specifically at the cleavage site by an enzyme, by cleavage light, or by a temperature change.

3. The connector according to claim 1, wherein the first reactive interactor and the second reactive interactor are configured to form a bioconjugate.

4. The connector according to claim 1, wherein first affinity interactor is one of streptavidin and biotin, and the second affinity interactor is another one of streptavidin and biotin.

5. The connector according to claim 4, wherein the streptavidin is a tetramer with one active biotin binding site, or a monomer with one active biotin binding site.

6. The connector according to claim 1, wherein the first affinity interactor is streptavidin, and the first affinity reagent is an oligonucleotide comprising biotin.

7. The connector according to claim 1, further comprising the first affinity reagent.

8. A marker for analysing biological samples, the marker comprising:
a label bound to the connector according to claim 7.

9. The marker according to claim 8, wherein the label comprises the second reactive interactor covalently bound to the first reactive interactor of the connector.

10. The marker according to claim 8, further comprising a third affinity reagent, the third affinity reagent comprising an oligonucleotide at least partially complementary to the first affinity reagent of the connector and hybridised to the first affinity reagent.

11. The marker according to claim 10, wherein the third affinity reagent further comprises an antibody.

12. The marker according to claim 9, wherein the label further comprises a base oligonucleotide, and at least one oligonucleotide conjugated dye hybridised to the base oligonucleotide via complementary base pairing.

13. A capture construct for capturing analytes of a biological sample, the capture construct comprising:
a DNA-origami backbone with at least a one capture region, and
at least one affinity capture reagent comprising the connector according to claim 7 and the second affinity reagent,
wherein the first affinity reagent of the connector binds the affinity capture reagent to the capture region of the DNA-origami backbone
wherein the first affinity reagent is a staple strand of the DNA-origami backbone, and
wherein the second affinity reagent is configured to capture one of the analytes of the biological sample.

14. A data storage device comprising:
a DNA-origami backbone with a plurality of attachment sites, and
a label bound to the connector according to claim 7,
wherein the first affinity reagent is an oligonucleotide staple strand of the DNA-origami backbone, and
wherein the first affinity reagent comprises a unique oligonucleotide sequence configured to bind the connector with the label to a complementary sequence of one of the plurality of attachment sites.

15. A method for generating the marker according to claim 8, the method comprising:
combining the first affinity reagent with the connector, and
combining the connector with the label.

16. The method according to claim 15, further comprising:
combining the first affinity reagent with a third affinity reagent.

17. A method for generating the capture construct according to claim 13, the method comprising:
combining the first affinity reagent with the connector,
combining the connector with the label or the second affinity reagent, and
combining the first affinity reagent with the DNA-origami backbone.

18. A device for generating a marker, the device comprising:
a liquid dispensing unit,
a plurality of liquid containers, and
a control unit configured to direct the liquid dispensing unit to dispense liquids containing labels, first affinity reagents, second affinity reagents, third affinity reagents, or DNA-origami backbones into the liquid containers according to the method of claim 15.

19. The device according to claim 18, wherein the liquid dispensing unit is configured to dispense the liquids with acoustic droplet ejection.

20. The capture construct according to claim 13, wherein the second affinity reagent comprises the second reactive interactor and is covalently bound to the first reactive interactor of the connector.
